(19) United States
(12) Patent Application Publication
(10) Pub. No.: US 2025/0332202 A1
(43) Pub. Date: Oct. 30, 2025

(54) LACTICASEIBACILLUS RHAMNOSUS ICL-46 STRAIN DERIVED FROM OAT AND ITS USE FOR ANTI-INFLAMMATION, ANTI-ALLERGY, SKIN IMMUNITY ENHANCEMENT, ATOPIC DERMATITIS IMPROVEMENT, SKIN SOOTHING, AND SKIN MICROORGANISM REGULATION

(71) Applicant: 360PERSPECTIVE INC, Seoul (KR)

(72) Inventors: Hwang Yeol LEE, Chungcheongbuk-do (KR); Ha Na CHO, Chungcheongbuk-do (KR); Christina BAE, Seoul (KR); Hyo Ryeon CHOI, Seoul (KR); Ye Na OH, Seoul (KR); Sol NAMGUNG, Seoul (KR); Hee Jin KIM, Seoul (KR); Young Joo OR, Seoul (KR)

(73) Assignee: 360PERSPECTIVE INC, Seoul (KR)

(21) Appl. No.: 19/001,562

(22) Filed: Dec. 26, 2024

(30) Foreign Application Priority Data
Dec. 27, 2023 (KR) 10-2023-0193106

(51) Int. Cl. A61K 35/744; A61K 8/99; A61P 17/00; A61P 29/00; A61P 37/08; A61Q 19/00; C12N 1/06; C12N 1/20; C12R 1/01

(52) U.S. Cl. CPC A61K 35/744; A61K 8/99; A61P 17/00; A61P 29/00; A61P 37/08; A61Q 19/00; C12N 1/066; C12N 1/205; A61K 2800/85; C12R 2001/01

(57) ABSTRACT

The present invention relates a novel Lacticaseibacillus rhamnosus ICL-46 strain and its uses for anti-inflammation, anti-allergic, skin soothing, and skin microorganism regulation. With the strain according to the aspect, it has anti-inflammation, anti-allergic, skin immunity enhancement, atopic dermatitis improvement, skin soothing, and skin microorganism regulating effects, so it can be applied in various ways to cosmetics, medicines, and foods.

### CROSS REFERENCE TO PRIOR APPLICATION

This application claims priority to Korean Patent Application No. 10-2023-0193106 filed on Dec. 27, 2023, which is hereby incorporated by reference in its entirety.

### SEQUENCE LISTING

This application contains a Sequence Listing submitted via EFS-Web and hereby incorporated by reference in its entirety. The Sequence Listing is named \2685-001.xml, created Dec. 19, 2024, and 3,450 bytes in size.

### BACKGROUND OF THE INVENTION

The present invention relates to a novel Avena sativa-derived microorganism and its use for anti-inflammation, anti-allergy, skin immunity enhancement, atopic dermatitis improvement, skin soothing, and skin microorganism regulation.

Probiotics are living microorganisms that provide healthy benefits. Representative strains of probiotics include Lactobacillus and Bifidobacterium family strains. Recently, research has been actively conducted to apply probiotic microorganisms to cosmetics to manufacture functional cosmetics.

Meanwhile, oats (Avena sativa) contains ingredients that are useful for the skin, and are widely used in the cosmetics field in the form of oat extract, oat oil, oat peptide, and oat kernel powder and the like.

However, research on oat-derived microorganisms and skin beauty effects thereof is not sufficient.

### SUMMARY OF THE INVENTION

One aspect provides a novel Lacticaseibacillus rhamnosus strain.

Another aspect provides a lysate, a culture medium, an extract of a culture medium, a fermentation medium, or any combination thereof of the said strain.

Another aspect provides a method for preparing a lysate of the strain.

Another aspect provides a composition comprising the Lacticaseibacillus rhamnosus strain, and a lysate, a culture medium, an extract of a culture medium, a fermentation medium, or any combination thereof of the said strain.

Another aspect provides use for anti-inflammation, anti-allergy, skin immunity enhancement, skin soothing, skin microorganism regulation use of the said composition.

Another aspect provides a method for preventing, improving, or treating a skin condition of a subject comprising administering an effective amount of the said composition to a subject in need thereof.

One aspect provides a Lacticaseibacillus rhamnosus ICL-46 strain.

The said strain may be isolated. The term “isolated” as used herein means that which is not present in nature but is artificially isolated and available. The said strain may be one isolated from oats (Oat or Avena sativa). The said strain may be one isolated from oat kernels, oat bran, or a mixture thereof.

The said strain may be a strain isolated by a method of inoculating oats into MRS medium and purely isolating and culturing the cultured colonies. As used herein, “Lacticazeibacillus rhamnosus isolated from oats” may be used interchangeably with “Lacticazeibacillus rhamnosus derived from oats”.

The said strain may be a strain belonging to the genus Lacticazeibacillus.

The said strain may comprise 16S rRNA having at least about 95%, at least about 96%, at least about 97%, at least about 98%, at least about 99%, at least about 99.5%, or at least about 99.9% a sequence identity with SEQ ID NO: 1. The said strain may be one that comprises 16S rRNA of SEQ ID NO: 1.

The term “sequence identity” as used herein refers to the degree of identity of amino acid residues or bases between sequences after aligning the two sequences to the maximum extent possible in a specific comparison region. The percentage of sequence identity can be determined using a known sequence comparison program, and examples thereof include BLASTN (NCBI), CLC Main Workbench (CLC bio), MegAlignTM (DNASTAR Inc), and the like.

The said Lacticazeibacillus rhamnosus strain may be a strain deposited under the accession number KCTC15702BP.

The said strain may have anti-inflammation, anti-allergic, skin immunity enhancement, skin soothing, or skin microorganism regulation effects.

Another aspect provides a lysate, a culture medium, an extract of a culture medium, a fermentation medium, or any combination thereof of the said strain.

The specific contents of the said strain are as described above.

The term “lysate” as used herein may mean one that comprises the intracellular biological components contained within a microorganism, for example, a lysate of useful components, such as a cell wall or membrane, and a culture containing metabolites released outside the cell during microbial culture. The said lysate may be prepared by pressure or ultrasonic shredding, excluding methods that induce modification of the metabolites of the microorganism, such as irradiation or heat inactivation. In one embodiment, the said lysate of the strain may be prepared by a microfluidizer (M.F.). Since the said useful components in the microorganism are easily deteriorated due to excessive temperature and energy, the lysate may be prepared using a microfluidizer, which can most stably obtain the useful components in the microorganism among the existing production processes.

The said lysate may include the lysate itself, a concentrate thereof, or a lyophilized product thereof.

As used herein, the term “culture medium” may be used interchangeably with “culture supernatant”, “conditioned culture medium” or “conditioned medium”, and may mean the entire medium including the said strain, its metabolites, extra nutrients, etc., obtained by culturing the said strain in a medium capable of supplying nutrients so that the Lacticazeibacillus rhamnosus strain can grow and survive in a test tube for a certain period of time. In addition, the said culture medium may mean a culture medium obtained by culturing the strain and removing the strain body from its culture medium. Meanwhile, the liquid from the culture medium from which the strain body have been removed is also called a “supernatant”, and may be obtained by allowing the culture medium to stand still for a certain period of time and taking only the liquid in the upper layer excluding the portion that has settled to the lower layer, or by removing the strain body through filtration, or by centrifuging the culture medium to remove the sediment at the lower layer and taking only the liquid at the upper layer. The said “strain body” means the strain itself of the present invention and includes the strain itself selected by separating from a sample and the like, or the strain isolated from the culture medium by culturing the said strain. The said strain body can be obtained by centrifuging the culture medium and taking the portion that has settled to the lower layer, or by allowing it to settle to the lower layer of the culture medium by gravity and then removing the upper liquid after leaving it alone for a certain period of time.

The said culture medium may include the culture medium itself obtained by culturing the strain, a concentrate or a lyophilized product thereof, or a culture supernatant obtained by removing the strain from the culture medium, a concentrate or a lyophilized product thereof.

The culture medium of the said strain can be separated into strain body and a supernatant by centrifugation. In the present specification, “lysate” may be a mixture of the culture medium of the strain and the strain body lysate.

The medium for culturing and culture conditions for culturing the said Lacticazeibacillus rhamnosus strain can be appropriately selected or modified and employed by a person of ordinary skill in the art. For example, the said medium may be a medium prepared with one selected from glucose, yeast extract, malt extract, peptone, glutamic acid, K₂HPO₄, KH₂PO₄, NaCl, MnSO₄, and MgSO⁴, or a mixture thereof. In addition, the said culture conditions may be, but are not limited to, culturing at a temperature of 30 to 40° C. for 12 to 72 hours.

The term “culture medium extract” in the present specification means an extract from the said culture medium or a concentrate thereof, and may include an extract, a dilution or concentrate of the extract, a dried product obtained by drying the extract, or a crude purified or purified product thereof, and a fraction obtained by fractionating the extract.

Another aspect provides a method for preparing a lysate of the said strain.

The said method may include a step of inoculating a Lacticazeibacillus rhamnosus strain according to one aspect into a medium and then culturing it to obtain a strain culture medium;
a step of centrifuging the said culture medium to separate the strain body and a supernatant; and
a step of mixing the lysate of the strain body and the supernatant to obtain a strain lysate.

Before the step of obtaining the said strain culture medium, a step of sterilizing the medium may be additionally included. The said sterilization may be performed according to a conventional method in the art, and may be performed at 110 to 130° C. for 1 minute to 1 hour, for example.

The said medium is not limited in type as long as it is a medium capable of culturing the Lacticazeibacillus rhamnosus strain. A medium for culturing and culture conditions for culturing the said Lacticazeibacillus rhamnosus strain can be appropriately selected or modified and employed by a person of ordinary skill in the art. For example, the said medium may be a medium prepared with one selected from glucose, yeast extract, malt extract, peptone, glutamic acid, K₂HPO₄, KH₂PO₄, NaCl, MnSO₄, and MgSO₄, or a mixture thereof. In addition, the said culture conditions may be, but are not limited to, culturing at a temperature of 30 to 40° C. for 12 to 72 hours. A strain culture medium may be obtained by the said culture.

In the step of centrifuging the culture medium to separate the strain body and the supernatant, a step of filtering after the centrifugation may be further included. The said filtration may be performed according to a conventional method in the art.

In the step of obtaining the said strain lysate, the said strain body may be lysed using a microfluidizer, but is not limited thereto.

Another aspect provides a use of the said Lacticazeibacillus rhamnosus strain, its lysate, culture medium, extract of the culture medium, fermentation medium, or any combination thereof. Specifically, a composition comprising a Lacticazeibacillus rhamnosus strain, a lysate thereof, a culture medium, an extract of a culture medium, a fermentation medium, or any combination thereof is provided.

In the said use or composition, the said Lacticazeibacillus rhamnosus strain may be a Lacticazeibacillus rhamnosus ICL-46 strain according to the said aspect.

The use of the said strain may include anti-inflammation, anti-allergy, skin immunity enhancement, skin soothing, skin microorganism regulation, and prevention or treatment of skin diseases.

The said strain may inhibit or reduce the expression of inflammation-related genes (e.g., COX-2, iNOS); inhibit or reduce the release of β-hexosaminidase, a major factor related to allergy or immunity; or inhibit or reduce skin harmful bacteria (e.g., P. aeruginosa, S. aureus) while not affecting skin beneficial bacteria (e.g., S. epidermidis).

The term “anti-inflammation” can be used interchangeably with “inflammation improvement” and “inflammation suppression” and can mean to any action that alleviates the immune response and inhibits the production of COX-2 or iNOS.

The term “anti-allergic” can be used interchangeably with “allergy improvement” and “allergy suppression” and can mean to any action that inhibits or improves the allergic response of the body.

The term “skin immunity enhancement” may mean any action that enhances the immunity of the skin, for example, but not limited to, by inhibiting an allergen (e.g., β-hexosaminidase) or inhibiting skin harmful bacteria.

The term “skin disease” may be a skin inflammatory disease, a skin allergic disease, or an immune skin disease. The term “prevention” includes inhibiting the occurrence of a disease. The term “treatment” includes inhibiting, alleviating, or eliminating the development of a disease.

The said skin inflammatory disease may be any one selected from the group consisting of skin wounds, dermatitis, atopic dermatitis, pruritus, eczematous skin disease, dry eczema, erythema, urticaria, psoriasis, rash, and acne.

The said skin allergic disease may be any one selected from the group consisting of atopic dermatitis, urticaria, rash, and pruritus.

The said immune skin disease is a general term for skin diseases related to immunity, and may include, for example, atopic dermatitis, seborrheic dermatitis, and psoriasis.

The term “skin soothing” means to any actions that calm and stabilize the heat or pain of irritated skin. The said skin soothing may include calming skin that has become sensitive due to inflammation, allergy, or weakened immunity.

The term “skin microorganism regulation” may mean to increasing beneficial skin bacteria and decreasing harmful skin bacteria; or suppressing or reducing only harmful skin bacteria without affecting beneficial skin bacteria. Specifically, skin microorganism regulation may mean suppressing or reducing the growth of harmful skin bacteria while not affecting the growth of beneficial skin bacteria. Beneficial skin bacteria and harmful skin bacteria may include all known strains. In one embodiment, the beneficial skin bacteria may be, but is not limited to, Staphylococcus epidermidis. The said skin harmful bacteria may be, but are not limited to, Pseudomonas aeruginosa or Staphylococcus aureus.

A composition according to one aspect may suppress or reduce the expression of at least one of COX-2 and iNOS by including the said Lacticaseibacillus rhamnosus strain, its lysate, culture medium, extract of culture medium, fermentation medium, or any combination thereof. Accordingly, the said composition may exhibit an anti-inflammation effect, a preventive or therapeutic effect on skin inflammation diseases.

A composition according to one aspect may suppress or reduce β-hexosaminidase, a major allergy factor, by including the said Lacticaseibacillus rhamnosus strain, its lysate, culture medium, extract of culture medium, fermentation medium, or any combination thereof. Therefore, the said composition can exhibit an anti-allergic effect, an effect of enhancing skin immunity, an effect of preventing or treating skin allergic diseases, and an effect of preventing or treating immune skin diseases.

The composition according to one aspect comprises the said Lacticazeibacillus rhamnosus strain, its lysate, culture medium, extract of culture medium, fermentation medium, or any combination thereof, thereby not affecting the growth of skin beneficial bacteria (S. epidermidis) but inhibiting or reducing the growth of skin harmful bacteria (P. aeruginosa, S. aureus). Therefore, the said composition can exhibit a skin microorganism regulation effect.

The composition according to one aspect can exhibit a skin soothing effect by at least one of the anti-inflammation, anti-allergy, skin immunity enhancement, and skin microorganism regulation effects.

In another embodiment, the said strain can be used together with another strain having a skin condition improvement effect to exhibit a synergistic effect.

The composition comprises 0.001 wt % to 80 wt %, for example, 0.01 wt % to 60 wt %, 0.01 wt % to 40 wt %, 0.01 wt % to 30 wt %, 0.01 wt % to 20 wt %, 0.01 wt % to 10 wt %, 0.01 wt % to 5 wt %, 0.05 wt % to 60 wt %, 0.05 wt % to 40 wt %, 0.05 wt % to 30 wt %, 0.05 wt % to 20 wt %, 0.05 wt % to 10 wt %, 0.05 wt % to 5 wt %, 0.1 wt % to 60 wt %, 0.1 wt % to 40 wt %, 0.1 wt % to 30 wt %, 0.1 wt % to 20%, 0.1 wt % to 10 wt %, or 0.1 wt % to 5 wt % of the strain, its lysate, culture medium, extract of the culture medium, fermentation medium, or any combination thereof based on the total weight of the composition.

The term “including as an effective ingredient” as used herein means that the strain, its lysate, culture medium, extract of the culture medium, fermentation medium, or any combination thereof of this specification is added to an extent that can exhibit the said effect, and includes formulation in various forms by adding various components as auxiliary components for drug delivery and stabilization and the like.

The said composition may be in a liquid or dry state. In one embodiment, the said composition may be in the form of a dry powder.

The drying method for manufacturing the composition in a dry state may use a method generally used in the art, and is not particularly limited. Non-limiting examples of the said drying method include an air drying method, a natural drying method, a spray drying method, a freeze drying method, and the like. These methods may be used alone or at least two methods may be used together.

The said composition may comprise an effective amount of an additive sufficient to reduce deterioration of the said strain, its lysate, a culture medium, an extract of the culture medium, a fermentation medium, or any combination thereof. The said additive may be, for example, but is not limited to, a binder.

The said composition may further include a cosmetically, pharmaceutically, or food-wise acceptable carrier. The said composition may be formulated with the carrier and provided as a cosmetic, drug, food additive, and the like.

The said composition may be a cosmetic composition.

In addition to the effective ingredients disclosed in the present, the said cosmetic composition may additionally include components commonly used in cosmetic compositions, functional additives, and the like, and may include conventional auxiliary agents such as antioxidants, stabilizers, solubilizers, surfactants, dispersants, preservatives, vitamins, pigments, fragrances, and the like, and a carrier.

The said cosmetic composition is not particularly limited to a specific formulation, and the formulation may be appropriately selected depending on the purpose. The said cosmetic composition may have, for example, a solubilized formulation, an emulsified formulation, or a dispersed formulation. The said cosmetic composition may have, for example, a cosmetic formulation of a flexible toner, a nourishing toner, a massage cream, a nourishing cream, an essence, a pack, a gel, an ampoule, or a skin adhesive type.

The said composition may be a composition for external skin application.

In the present specification, the external skin application may be a cream, a gel, an ointment, a skin emulsifier, a skin suspension, a transdermal delivery patch, a drug-containing bandage, a lotion, or any combination thereof. The said external skin application may be appropriately mixed with ingredients commonly used in external skin applications such as cosmetics or medicines, for example, aqueous ingredients, oily ingredients, powder ingredients, alcohols, moisturizers, thickeners, ultraviolet absorbers, whitening agents, preservatives, antioxidants, surfactants, fragrances, colorants, various skin nutrients, or any combination thereof, as needed.

The said composition may be a pharmaceutical composition.

The said pharmaceutical composition may additionally include a pharmaceutically acceptable diluent or carrier. The said diluent may be lactose, corn starch, soybean oil, microcrystalline cellulose, mannitol, or any combination thereof. The said carrier may be an excipient, a disintegrant, a binder, a glidant, or any combination thereof. The said excipient may be microcrystalline cellulose, lactose, a low-substituted hydroxycellulose, or any combination thereof. The said disintegrant may be calcium carboxymethylcellulose, sodium starch glycolate, calcium dihydrogen phosphate, or any combination thereof. The said binder may be polyvinylpyrrolidone, a low-substituted hydroxypropylcellulose, hydroxypropylcellulose, or any combination thereof. The said glidant may be magnesium stearate, silicon dioxide, talc, or any combination thereof.

The said pharmaceutical composition may be formulated as an oral or parenteral administration form. Oral administration forms may include granules, powders, liquids, tablets, capsules, dry syrups, and the like. Parenteral administration forms may include injections, ointments, and the like.

The said composition may be a food composition. In this case, it may be formulated as a formulation of a conventional health functional food known in the art. Accordingly, the said food composition may be a health functional food composition.

The said food composition may be used alone or in combination with other foods or food ingredients, using the said strain, its lysate, culture medium, extract of culture medium, fermentation medium, or any combination thereof, and may be appropriately used according to a conventional method. The mixed amount of the effective ingredient may be suitably determined depending on the purpose of use (prevention, health, or therapeutic treatment). There is no particular limitation on the type of the said health functional food. Among the types of health functional foods, the beverage composition may contain various flavoring agents or natural carbohydrates as additional ingredients, like a conventional beverage. The said natural carbohydrates include monosaccharides such as glucose and fructose; disaccharides such as maltose and sucrose; and polysaccharides such as dextrin and cyclodextrin; and sugar alcohols such as xylitol, sorbitol, and erythritol. As a sweetener, a natural sweetener such as thaumatin, stevia extract, or a synthetic sweetener such as saccharin, aspartame, and the like can be used. The said food composition may also contain a nutrient, a vitamin, an electrolyte, a flavoring agent, a coloring agent, pectic acid and its salts, alginic acid and its salts, an organic acid, a protective colloid thickener, a pH adjusting agent, a stabilizer, a preservative, glycerin, alcohol, a carbonating agent used in carbonated beverages, or any combination thereof. The said food composition may also contain fruit pulp for the production of natural fruit juice, fruit juice beverage, vegetable beverage, or any combination thereof.

Another aspect provides a method for preventing, improving, or treating a skin condition of a subject, comprising administering an effective amount of the said composition to a subject in need thereof.

The said skin condition of the subject may be a condition related to inflammation, allergy, or immunity.

The terms “administering,” “introducing,” and “implanting” are used interchangeably herein and may refer to placement of a composition according to an embodiment into a subject by a method or route that results in at least partial localization to a desired site of the composition according to an embodiment.

Administration may be by any method known in the art. Administration may be by any means, for example, intravenous, intramuscular, oral, transdermal, mucosal, intranasal, intratracheal, or subcutaneous administration, directly into the subject. The said administration may be systemic or local.

The said subject may be a mammal, for example, a human, a cow, a horse, a pig, a dog, a sheep, a goat, or a cat. The said subject may be a subject in need of improvement in skin condition, for example, an anti-inflammation, anti-allergic, skin immune enhancement, skin soothing, or skin microorganism modulating effect.

The said administration may be conducted by dosing 0.1 mg to 1,000 mg per day per individual of the composition according to one embodiment, for example, 0.1 mg to 500 mg, 0.1 mg to 100 mg, 0.1 mg to 50 mg, 0.1 mg to 25 mg, 1 mg to 1,000 mg, 1 mg to 500 mg, 1 mg to 100 mg, 1 mg to 50 mg, 1 mg to 25 mg, 5 mg to 1,000 mg, 5 mg to 500 mg, 5 mg to 100 mg, 5 mg to 50 mg, 5 mg to 25 mg, 10 mg to 1,000 mg, 10 mg to 500 mg, 10 mg to 100 mg, 10 mg to 50 mg, or 10 mg to 25 mg. However, the dosage may be prescribed in various ways depending on factors such as the formulation method, administration method, patient's age, weight, sex, pathological condition, food, administration time, administration route, excretion rate, and response sensitivity, and a person skilled in the art can appropriately adjust the dosage by considering these factors. The number of administrations can be once a day or twice or more within the range of clinically acceptable side effects, and the administration can be conducted to one or two or more sites, and the total number of administration days can be from 1 day to 30 days for one treatment, daily or at intervals of 2 to 5 days. If necessary, the same treatment can be repeated after an appropriate period. For animals other than humans, the same dosage as for humans per kg can be administered, or the amount converted from the said dosage can be administered based on the volume ratio (e.g., average value) of the organs (heart, etc.) of the target animal and the human, for example.

With the novel Lacticaseibacillus rhamnosus strain according to one aspect, it has anti-inflammatory, anti-allergy, skin immunity enhancement, atopic dermatitis improvement, skin soothing, or skin microorganism regulation effects, and therefore can be applied in various ways to cosmetics, pharmaceuticals, and foods.

### BRIEF DESCRIPTION OF THE DRAWINGS

FIG. 1 shows the results showing the COX-2 mRNA expression level according to the class of oat-derived lactic acid bacteria strain.

FIG. 2 shows the results showing the iNOS mRNA expression level according to the class of oat-derived lactic acid bacteria strain.

FIG. 3 shows the results showing the β-hexosaminidase inhibition effect according to the class of oat-derived lactic acid bacteria strain.

FIG. 4 shows the results showing the growth degree according to the treatment of Lacticazeibacillus rhamnosus ICL-46 lysate when co-cultivating the skin beneficial bacteria S. epidermidis and the harmful bacteria P. aeruginosa.

FIG. 5 shows the results showing the growth degree according to the treatment of Lacticazeibacillus rhamnosus ICL-46 lysate when co-cultivating the skin beneficial bacteria S. epidermidis and the harmful bacteria S. aureus.

### DETAILED DESCRIPTION OF THE INVENTION

Hereinafter, the present invention will be described in more detail through examples. However, these examples are intended to exemplify the present invention and the scope of the present invention is not limited to these examples.

### Example 1. Isolation and Identification of Strains Derived From Oats

Isolation and identification of microorganisms from oats were performed.

Specifically, undried oat kernels and oat bran were crushed using a sterilized grinder. 10 g of the crushed material was added to 90 mL of MRS (de Man, Rogosa and Sharpe) liquid medium and cultured at 37° C. for about 48 hours.

The cultured liquid medium was diluted decimal in saline solution and plated on an MRS plate and cultured for more than 48 hours under the same culture conditions. Thereafter, strains were randomly isolated from the generated colonies.

Randomly isolated strains were subcultured more than three times, and the isolated pure colonies were loaded on BCP (Bromocresol Purple) Agar Plate (Manufacturer: Eiken Chemical, Product Name: BCP Plate Count Agar) to confirm the acid-producing ability. The strains with milky white colonies and yellow surroundings were selected. The selected colonies were analyzed for the base sequence of the 16S rRNA gene for identification and classification.

As a result of performing a similarity search on NCBI for the analyzed 16S rRNA base sequence, it showed 99.67% homology with Lacticaseibacillus rhamnosus.

A novel microorganism Lacticaseibacillus rhamnosus strain (named “ICL-46”) isolated from the said oats was deposited with the Korea Research Institute of Bioscience and Biotechnology (KCTC) on Nov. 20, 2023 and assigned the accession number KCTC15702BP. The ICL-46 strain has the 16S IRNA sequence of SEQ ID NO: 1 (complementary DNA).

### Example 2. Preparation of Lacticaseibacillus Rhamnosus ICL-46 Lysate

The medium was sterilized at 121° C. for 15 minutes. The medium can be appropriately selected according to the class of microorganism to be inoculated, and for example, a medium prepared with one selected from glucose, yeast extract, malt extract, peptone, glutamic acid, K₂HPO₄, KH₂PO₄, NaCl, MnSO₄, and MgSO₄ or a mixture thereof can be used.

After inoculating the Lacticazeibacillus rhamnosus ICL-46 strain of Example 1 into the sterilized medium, it was cultured for 24 hours at 37° C.

After the culture was completed, the strain body and supernatant were separated by centrifugation. The strain body were disrupted by passing them twice at 800 bar using a microfluidizer. The disrupted strain body and the said supernatant were mixed to obtain the lysate of the Lacticazeibacillus rhamnosus ICL-46 strain.

### Comparative Examples 1 to 4. Isolation of Lactic Acid Bacteria Derived From Oats and Preparation of a Lysate

Lactic acid bacteria were isolated from oats using the same method as in the said Example 1. Through base sequence analysis of the 16S rRNA gene, Lactiplantibacillus plantarum strain, Lactococcus lactis strain, Latilactobacillus sakei strain, and Lacticaseibacillus rhamnosus strain were identified. The Lacticaseibacillus rhamnosus strain was identical in species to the ICL-46 strain of Example 1, but had a difference in the 16S rRNA sequence.

The lysates of each of the said oat-derived lactic acid bacteria were prepared in the same manner as in the said Example 2. The lysate of the Lactiplantibacillus plantarum strain was Comparative Example 1, the lysate of the Lactococcus lactis strain was Comparative Example 2, the lysate of the Latilactobacillus sakei strain was Comparative Example 3, and the lysate of the Lacticazeibacillus rhamnosus strain was Comparative Example 4.

### Experimental Example 1. Confirmation of Anti-Inflammation Efficacy—Expression Analysis of COX-2 and iNOS

In order to confirm the anti-inflammation effect of the lysate of the Lacticazeibacillus rhamnosus ICL-46 strain, the expression of COX-2 (cyclooxygenase-2) and iNOS (Nitric oxide synthases) genes was analyzed through Real-Time PCR.

Specifically, after LPS (liposaccharides) was treated on mouse macrophage Raw 264.7, 0.5% (v/v) of the strain lysates of Example 2 and Comparative Examples 1 to 4 was added, and the expression levels of COX-2 and iNOS genes were confirmed. The group that was not treated with anything (MO: media only) was used as a negative control group, and the group that was treated only with LPS was used as a positive control group.

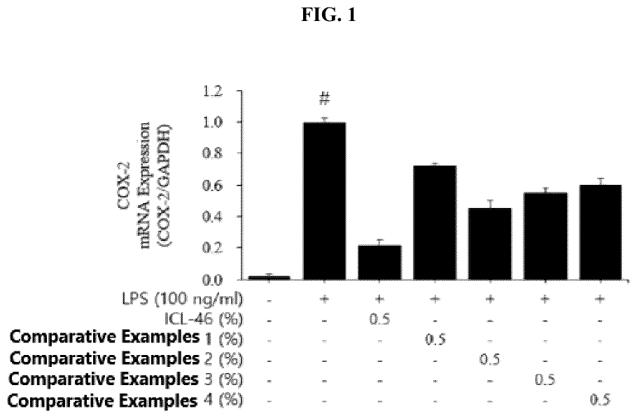

FIG. 1 shows the results showing the level of COX-2 mRNA expression according to the class of oat-derived lactic acid bacteria strain.

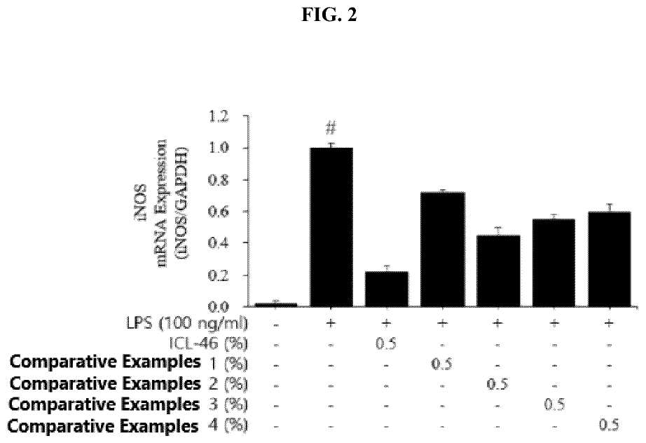

FIG. 2 shows the results showing the level of iNOS mRNA expression according to the class of oat-derived lactic acid bacteria strain.

As a result, as shown in FIGS. 1 and 2, it was confirmed that the lysate of the Lacticazeibacillus rhamnosus ICL-46 strain had the best effect on inhibiting COX-2 and iNOS gene expression compared to other oat-derived lactic acid bacteria strains.

From the above results, it could be confirmed that the lysate of the Lacticazeibacillus rhamnosus ICL-46 strain exhibited a skin soothing effect through a remarkably excellent anti-inflammation effect.

### Experimental Example 2. Confirmation of Anti-Allergic Efficacy—Inhibition of β-Hexosaminidase Release

In order to confirm the anti-allergic effect of the lysate of the Lacticazeibacillus rhamnosus ICL-46 strain, the effect of inhibiting the release of β-hexosaminidase, a major factor related to allergy or immunity, was tested. Inhibition of β-hexosaminidase release means preventing degranulation of immune cells.

Specifically, RBL-2H3 cells were equally divided into 24-well plates at 2X10⁵ cells/well and stabilized for 24 hours under conditions of 37° C. and 5% CO2. After washing the cells with PBS, each well was treated with anti-DNP-IgE (50 ng/ml) and cultured for 24 h. The cells sensitized with anti-DNP-IgE were washed twice with MEM medium supplemented with 1% FBS, 160 μL of the said medium was added, and cultured at 37° C. for 20 minutes. Thereafter, 0.5% (v/v) of each of the strain lysates of Example 2 and Comparative Examples 1 to 4 was added, and 20 μL of DNP-BSA having a concentration of 1 μg/mL was added and reacted at 37° C. for 1 h to allow the cells to form granules, and the supernatant was centrifuged at 4° C., 13,000 rpm, and for 10 minutes. 50 μL of 0.1 M citrate buffer (10 mM p-nitrophenyl-N-acetyl-β-D-glucosaminide, pH 4.5) was added to 25 μL of the centrifuged supernatant, and the reaction was performed at 37° C. for 1 h. After that, 100 μL of stop buffer (0.1 M Na₂CO₃/NaHCO₃, pH 10.0) was added to stop the reaction, and the absorbance was measured at 405 nm using a microplate reader.

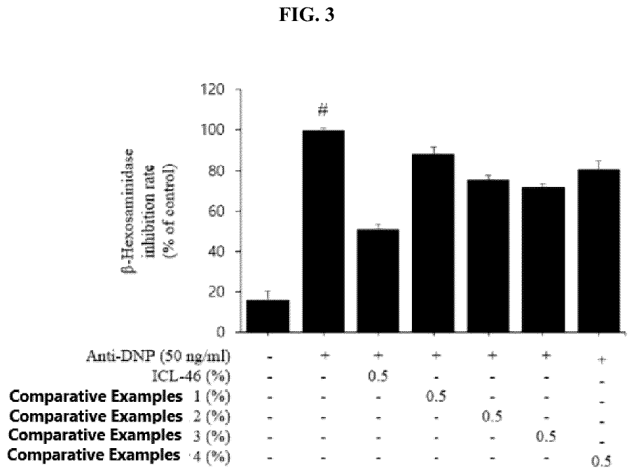

FIG. 3 shows the results showing the β-hexosaminidase inhibition effect according to the class of oat-derived lactic acid bacteria strain.

As a result, as shown in FIG. 3, it was confirmed that the lysate of the Lacticazeibacillus rhamnosus ICL-46 strain had the best effect on reducing β-hexosaminidase activity compared to other oat-derived lactic acid bacteria strains.

From the above results, it could be confirmed that the Lacticazeibacillus rhamnosus ICL-46 lysate exhibited significantly excellent anti-allergic or skin immunity enhancing efficacy.

### Experimental Example 3. Confirmation of SKIN MICROORGANISM REGULATION EFFECT

An experiment was conducted to confirm the skin microorganism regulation effect of the lysate of the Lacticazeibacillus rhamnosus ICL-46 strain, which was confirmed to have the best anti-inflammation and anti-allergic effects through Experimental Examples 1 and 2.

Specifically, TSB (tryptic soy broth, BD Difco, USA) was used as the microbial medium. The growth of the bacteria was compared by inoculating the corresponding strain into the appropriate medium at a concentration of 1X10⁶ CFU/mL and culturing it at 37° C. for 24 h, and then measuring the absorbance at 600 nm, or the growth rate was compared by confirming the actual number of viable bacteria through the serial dilution method. The percentage comparison was made based on the experimental group to which the same amount of phosphate buffered saline as the test sample was added.

1 mL of 10% TSB was added to a 12-well plate for co-culture of harmful and beneficial skin bacteria, and harmful bacteria (Pseudomonas aeruginosa, Staphylococcus aureus) were inoculated at a concentration of 1X10⁶ CFU/mL. A 0.4 um cell culture insert was placed on top, and beneficial bacteria (Staphylococcus epidermidis) were inoculated at a concentration of 1X10⁶ CFU/mL in 1 mL of 10% TSB. The 10% TSB was treated with 0.5% (v/v) of the lysate of Lacticazeibacillus rhamnosus ICL-46 of Example 2, and then the experiment was conducted. The same amount of phosphate buffered saline was treated as the control group. Subsequently. after culturing at 32° C. for 12 h, the number of each bacteria was checked and the degree of growth was compared.

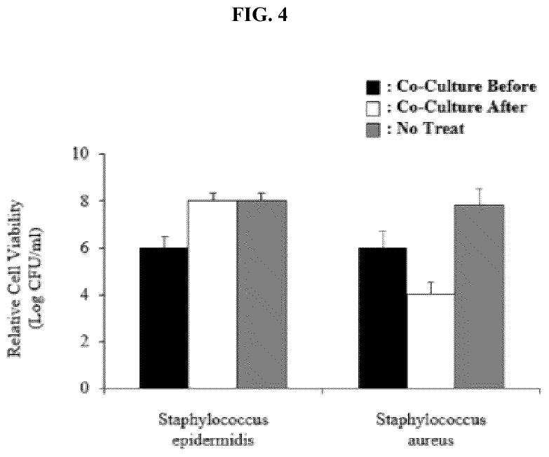

FIG. 4 shows the results of comparing the growth degree according to the treatment of Lacticazeibacillus rhamnosus ICL-46 lysate when co-cultivating the skin beneficial bacteria S. epidermidis and the harmful bacteria P. aeruginosa.

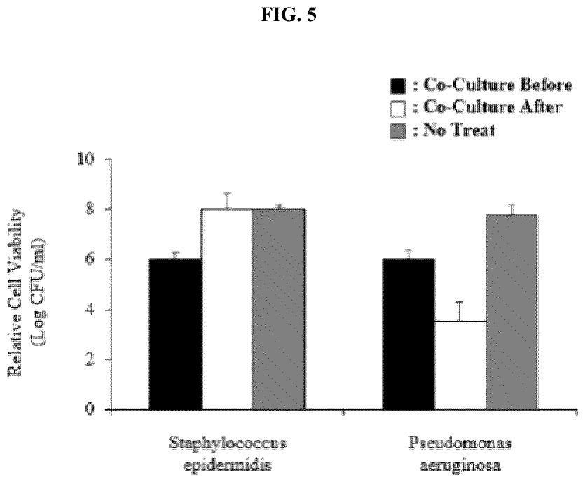

FIG. 5 shows the results of comparing the growth degree according to the treatment of Lacticazeibacillus rhamnosus ICL-46 lysate when co-cultivating the skin beneficial bacteria S. epidermidis and the harmful bacteria S. aureus.

As a result, as shown in FIGS. 4 and 5, it was confirmed that when the Lacticaseibacillus rhamnosus ICL-46 lysate was treated, the growth of the beneficial bacteria (S. epidermidis) was not affected, while the growth of the harmful bacteria (P. aeruginosa, S. aureus) was inhibited or reduced.

From the above results, it could be confirmed that the Lacticaseibacillus rhamnosus ICL-46 lysate exhibited significantly excellent skin microorganism regulation efficacy.

In summary, the oat-derived Lacticaseibacillus rhamnosus ICL-46 strain inhibited or reduced the expression of COX-2 and iNOS; inhibited or reduced the release of β-hexosaminidase, a major factor related to allergy or immunity; and did not affect the growth of skin beneficial bacteria (S. epidermidis), while reducing the growth of skin harmful bacteria (P. aeruginosa, S. aureus). Therefore, it was found that the oat-derived Lacticaseibacillus rhamnosus ICL-46 strain had skin inflammation improvement (anti-inflammation) effects, skin allergy improvement (anti-allergic) effects, skin immunity enhancement effects, skin soothing effects, and skin microorganism regulation effects.

The above description is merely an illustrative description of the technical idea of the present invention, and those skilled in the art will appreciate that various modifications and variations may be made without departing from the essential characteristics of the present invention.

Name of depositor: Korea Research Institute of Bioscience and Biotechnology (KRIBB), Deposit date: Nov. 20, 2023, Accession number: KCTC15702BP, the address of the KRIBB is 125 Gwahak-ro, Yuseong-gu, Daejeon 334141, Republic of Korea. The deposit was made under Budapest Treaty, and that all restrictions imposed by the depository will be irrevocably removed upon the granting of the patent. The deposit is hereby incorporated by reference in its entirety.

What is claimed is:

1. Lacticaseibacillus rhamnosus ICL-46 strain deposited under the accession number KCTC15702BP.

2. The strain of claim 1, which is isolated from oat (Avena sativa).

3. The strain of claim 1, which comprises 16S rRNA of SEQ ID NO: 1.

4. A method for preparing a lysate of the strain of claim 1, comprising the steps of inoculating the strain of claim 1 into a medium and then culturing it to obtain a strain culture medium;
a step of centrifuging the said culture medium to separate the strain body and a supernatant; and
a step of mixing the said lysate of the strain body and the supernatant to obtain a strain lysate.

5. A method of improving skin inflammation, improving skin allergies, promoting skin immunity, or soothing skin, comprising:
administering a cosmetic compositing comprising at least one of the strain of claim 1, its lysate, culture medium, extract of culture medium, and fermentation medium to a subject.

6. A method of regulating skin microorganisms, comprising:
administering a cosmetic composition comprising at least one of the strain of claim 1, its lysate, culture medium, extract of culture medium, and fermentation medium to a subject.

7. The method of claim 6, wherein the skin microorganism regulation suppresses the growth of skin harmful bacteria.

8. The method of claim 7, wherein the skin harmful bacteria is Pseudomonas aeruginosa or Staphylococcus aureus.

9. A method of preventing or treating skin inflammation, skin allergy, or immune skin disease, comprising:
administering a pharmaceutical composition comprising at least one of the strain of claim 1, its lysate, culture medium, extract of culture medium, and fermentation medium to a subject.

10. The method of claim 9, the pharmaceutical composition inhibits or reduces the expression of at least one of COX-2 (cyclooxygenase-2) and iNOS (Nitric oxide synthases); or inhibits or reduces the release of β-hexosaminidase.

11. A method of improving skin inflammation, improving skin allergies, promoting skin immunity, or soothing skin, comprising:
administering a health functional food composition comprising at least one of the strain of claim 1, its lysate, culture medium, extract of culture medium, and fermentation medium to a subject.


SEQUENCE LISTING (1 sequence)

SEQ ID NO: 1 - DNA, 1445 bp, Lacticaseibacillus rhamnosus
  source 1..1445: mol_type = other DNA
gcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttctagggagcgagccgtctaaggtg